(19) United States
(12) Patent Application Publication
(10) Pub. No.: US 2018/0251807 A1
(43) Pub. Date: Sep. 6, 2018

(54) Method for Fermenting Rosa sterilis var. leioclada

(71) Applicant: JIAN MAO BIOTECH CO., LTD., Kaohsiung (TW)

(72) Inventors: Yi-Jinn Chen, Kaohsiung (TW); Chang-Lu Hsu, Kaohsiung (TW); Jung-Lung Lo, Kaohsiung (TW); Shui-Chen Huang, Kaohsiung (TW); Hui-Ting Chan, Kaohsiung (TW)

(21) Appl. No.: 15/843,779

(22) Filed: Dec. 15, 2017

(30) Foreign Application Priority Data
Mar. 3, 2017 (TW) 106107074

(51) Int. Cl. C12P 39/00; C12G 3/02; A23L 19/00; A23L 33/00

(52) U.S. Cl. CPC C12P 39/00; A23L 33/00; A23L 19/00; C12G 3/02

(57) ABSTRACT

A method for fermenting Rosa sterilis var. leioclada includes providing a fruit sample of Rosa sterilis var. leioclada. A Lactobacillus casei strain, a Bifidobacterium longum strain and a Saccharomyces cerevisiae strain are mixed in a cell number ratio of 0.3-1:0.2-0.9:1-1.8 to form a microbial mixture. The fruit sample of Rosa sterilis var. leioclada is then fermented by the microbial mixture at 22-33° C. for 6-15 days to obtain a fermented juice. A total concentration of the Lactobacillus casei strain, the Bifidobacterium longum strain and the Saccharomyces cerevisiae strain in the mixture solution containing the microbial mixture and the fruit sample of Rosa sterilis var. leioclada is between 1.5×10⁷ and 3.5×10⁷ CFU/mL.

### CROSS REFERENCE TO RELATED APPLICATIONS

The application claims the benefit of Taiwan application serial No. 106107074, filed on Mar. 3, 2017, and the entire contents of which are incorporated herein by reference.

### BACKGROUND OF THE INVENTION

### 1. Field of the Invention

The present invention generally relates to a method for fermenting Rosa sterilis var. leioclada and, more particularly, to a method for fermenting Rosa sterilis var. leioclada to obtain a fermented juice with improved anti-oxidation ability.

### 2. Description of the Related Art

As the industrialization progresses, more and more pollutants are found in the environment. People come into intact with the pollutants every day, resulting in free radicals found in the human body, which are the main causes of many civilized diseases in modern times. Therefore, people nowadays pay more and more attention to anti-oxidation to remove the free radicals found in the human bodies.

Taking fruits of Rosa sterilis var. leioclada (Rosaceae) is a way to absorb antioxidants. However, in order to obtain enough antioxidants, a lot of fruits should be taken. In light of this, it is necessary to develop a method for fermenting Rosa sterilis var. leioclada to obtain a fermented juice with improved anti-oxidation ability.

### SUMMARY OF THE INVENTION

It is therefore the objective of the present invention to provide a method for fermenting Rosa sterilis var. leioclada to obtain a fermented juice with improved anti-oxidation ability.

One embodiment of the invention discloses a method for fermenting Rosa sterilis var. leioclada. The method includes providing a fruit sample of Rosa sterilis var. leioclada. A Lactobacillus casei strain, a Bifidobacterium longum strain and a Saccharomyces cerevisiae strain are mixed in a cell number of 0.3-1:0.2-0.9:1-1.8 to form a microbial mixture. The fruit sample of Rosa sterilis var. Leioclada is fermented by the microbial mixture at 22-33° C. for 6-15 days to obtain a fermented juice. A total concentration of the L. casei strain, the B. longum strain and S. cerevisiae strain in a mixture solution containing the microbial mixture and the fruit sample of Rosa sterilis var. leioclada is between 1.5×10⁷ and 3.5×10⁷ CFU/mL.

Accordingly, by fermenting the fruit sample by the microbial mixture including the L. casei strain, the B. longum strain and the S. cerevisiae strain, the obtained fermented juice has improved anti-oxidation ability. Therefore, the fermented juice can be used as a healthy product as well as an additive with anti-oxidation ability.

In an example, a Lactobacillus casei strain BCRC 10697, a Bifidobacterium longum strain BCRC 14604 and a Saccharomyces cerevisiae strain BCRC 20579 are mixed to form the microbial mixture.

In an example, the fruit sample of Rosa sterilis var. leioclada is fermented by the microbial mixture at 28° C.

In an example, the fruit sample of Rosa sterilis var. leioclada is fermented by the microbial mixture for 14 days.

### BRIEF DESCRIPTION OF THE DRAWINGS

FIG. 1a depicts a bar chart illustrating ABTS+ clearance rate of the fermented juices of groups A1 & A2, as well as the unfermented juice of group A3 in trial (A).

FIG. 1b depicts a bar chart illustrating SOD activity of the fermented juices of groups A1 & A2, as well as the unfermented juice of group A3 in trial (A).

FIG. 1c depicts a bar chart illustrating vitamin C level of the fermented juices of groups A1 & A2, as well as the unfermented juice of group A3 in trial (A).

FIG. 1d depicts a bar chart illustrating carotenoid level of the fermented juices of groups A1 & A2, as well as the unfermented juice of group A3 in trial (A).

FIG. 2a depicts a bar chart illustrating ABTS+ clearance rate of the fermented juices of groups B1 & B2, as well as the unfermented juice of group B3 in trial (B).

FIG. 2b depicts a bar chart illustrating SOD activity of the fermented juices of groups B1 & B2, as well as the unfermented juice of group B3 in trial (B).

FIG. 2c depicts a bar chart illustrating vitamin C level of the fermented juices of groups B1 & B2, as well as the unfermented juice of group B3 in trial (B).

FIG. 2d depicts a bar chart illustrating carotenoid level of the fermented juices of groups B1 & B2, as well as the unfermented juice of group B3 in trial (B).

FIG. 3a depicts a bar chart illustrating ABTS+ clearance rate of the fermented juices of groups C1 & C2, as well as the unfermented juice of group C3 in trial (C).

FIG. 3b depicts a bar chart illustrating SOD activity of the fermented juices of groups C1 & C2, as well as the unfermented juice of group C3 in trial (C).

FIG. 3c depicts a bar chart illustrating vitamin C level of the fermented juices of groups C1 & C2, as well as the unfermented juice of group C3 in trial (C).

FIG. 3d depicts a bar chart illustrating carotenoid level of the fermented juices of groups C1 & C2, as well as the unfermented juice of group C3 in trial (C).

FIG. 4a depicts a bar chart illustrating ABTS+ clearance rate of the fermented juices of groups D1 & D2, as well as the unfermented juice of group D3 in trial (D).

FIG. 4b depicts a bar chart illustrating SOD activity of the fermented juices of groups D1 & D2, as well as the unfermented juice of group D3 in trial (D).

FIG. 4c depicts a bar chart illustrating vitamin C level of the fermented juices of groups D1 & D2, as well as the unfermented juice of group D3 in trial (D).

FIG. 4d depicts a bar chart illustrating carotenoid level of the fermented juices of groups D1 & D2, as well as the unfermented juice of group D3 in trial (D).

### DETAILED DESCRIPTION OF THE INVENTION

A method for fermenting Rosa sterilis var. leioclada according to an embodiment of the present invention approximately includes providing a fruit sample of Rosa sterilis var. leioclada, abbreviated as “the fruit sample”. A microbial mixture then is used to ferment the fruit sample, obtaining a fermented juice of Rosa sterilis var. leioclada, abbreviated as “the fermented juice”, with improved anti-oxidation ability. Moreover, the microbial mixture includes a L. casei strain, a B. longum strain and a S. cerevisiae strain.

Specifically, a L. casei strain BCRC 10697, a B. longum strain BCRC 14604 and a S. cerevisiae strain BCRC 20579, purchased from the Bioresource Collection and Research Center (BCRC) of the Food Industry Research and Development Institute (FIRDI) of Taiwan, can be used to form the microbial mixture. Alternatively, the L. casei strain BCRC 10697, the B. longum strain BCRC 14604 and the S. cerevisiae strain BCRC 20579 can also be purchased from American Type Culture Collection (ATCC) of the United States with the number of ATCC 393, ATCC 15702 and ATCC 2366, respectively.

The fruit sample can be a whole fruit with pulp, peel and seeds. As an example, the whole fruit can be steamed and softened, and the microbial mixture can infiltrate into the whole fruit and fermentation efficiency can be effectively increased. Alternatively, the fruit sample can be the whole fruit which is incised, such as the whole fruit with an incision formed on the peel of the whole fruit or the whole fruit which is cut in half or sliced into several pieces. Therefore, pulp of Rosa sterilis var. leioclada is exposed to the microbial mixture and fermentation efficiency can be effectively increased. In this embodiment, the fruit sample is selected to be a juice of Rosa sterilis var. leioclada, abbreviated as “the juice”, which is obtained by squeezing the whole fruit (with pulp, peel and seeds). Accordingly, compared to using the whole fruit (with pulp, peel and seeds) and the whole fruit which is incised as the fruit sample, using the juice as the fruit sample increases mixing efficiency of the fruit sample and the microbial mixture, as well as the fermentation efficiency.

To activate the L. casei strain, the B. longum strain and the S. cerevisiae strain, the L. casei strain, the B. longum strain and the S. cerevisiae strain can preferably be cultured before the fruit sample is fermented by the microbial mixture. With such performance, the L. casei strain, the B. longum strain and the S. cerevisiae strain can become healthier and the fermentation efficiency can thus be improved. In this embodiment, the L. casei strain, the B. longum strain and the S. cerevisiae strain are cultured in a liquid medium separately to prevent from inhibition of growth of one strain due to growth of another strain. The L. casei strain, the B. longum strain and the S. cerevisiae strain are cultured to log phase (logarithmic phase) and a solution of L. casei, a solution of B. longum and a solution of S. cerevisiae are formed respectively. Moreover, the L. casei strain, the B. longum strain and the S. cerevisiae strain can be separately cultured at 37° C. which is the optimal culturing temperature for all of the L. casei strain, the B. longum strain and the S. cerevisiae strain, and thus quality of the L. casei strain, the B. longum strain and the S. cerevisiae strain can be assured. Besides, cell number of the L. casei strain, the B. longum strain and the S. cerevisiae strain can be counted using a hemocytometer, and thus concentrations of the solution of L. casei, the solution of B. longum and the solution of S. cerevisiae can be adjusted to 1×10⁷ CFU/mL. It is worthy to noted that in the microbial mixture, the cell number ratio of the L. casei strain, the B. longum strain and the S. cerevisiae strain is 0.3-1:0.2-0.9:1-1.8, preferably the cell number percentages of the L. casei strain, the B. longum strain and the S. cerevisiae strain is 20-25%, 13-25% and 50-64%, respectively. Thus, the L. casei strain, the B. longum strain and the S. cerevisiae strain can have great interaction efficiency. Preferably, the cell number ratio of the L. casei strain, the B. longum strain and the S. cerevisiae strain is 0.8:0.8:1.6 to form the microbial mixture. That is, the cell number percentages of the L. casei strain, the B. longum strain and the S. cerevisiae strain are 25%, 25% and 50% by cell number of the microbial mixture, respectively. In such performance, the best fermentation efficiency can be achieved.

Moreover, the microbial mixture and the fruit sample can be mixed to obtain a mixture solution. A total concentration of the L. casei strain, the B. longum strain and the S. cerevisiae strain in the mixture solution is between 1.5×10⁷ and 3.5×10⁷ CFU/mL. In addition, a volume percentage of the microbial mixture can be 10-25% by volume of the mixture solution. Accordingly, the fermentation process can be processed with enough microbial cells and the fermentation efficiency can be assured.

The fruit sample is then fermented by the microbial mixture at the optimal condition to obtain the fermented juice. In detail, the fruit sample is fermented by the microbial mixture at 22-33° C. for 6-15 days. Moreover, the fruit sample can be fermented by the microbial mixture with or without agitation. As an example, the fruit sample can be fermented by the microbial mixture with agitation at a rotation speed of 80-110 rpm to prevent from the occurrence of precipitation. In this embodiment, the fruit sample is fermented by the microbial mixture at 28° C. for 14 days with agitation at the rotation speed of 100 rpm.

To evaluate the fermented juice with improved anti-oxidation ability can be obtained by using the method for fermenting Rosa sterilis var. leioclada according to the present invention, the following trials are carried out.

Trial (A).

In trial (A), the L. casei strain, the B. longum strain and the S. cerevisiae strain are mixed in the cell number ratio of 0.8:0.8:1.6 to obtain the microbial mixture. The juice is then fermented by the microbial mixture at 28° C. for 14 days to form the fermented juice of group A1. Moreover, a Lactobacillus acidophilus strain, the B. longum strain and the S. cerevisiae strain are mixed in a cell number ratio of 0.8:0.8:1.6 to obtain another microbial mixture. The juice is then fermented by the another microbial mixture at 28° C. for 14 days to form the fermented juice of group A2. An unfermented juice is used as group A3.

ABTS (2,2′-Azino-bis-[3-ethylbenthiazoline sulfonic acid]) is a chromogenic agent. ABTS becomes ABTS+, a stable green substance absorbing light at 734 nm, when interacts with an oxidant in the presence of hydrogen peroxide (H₂O₂). Moreover, the presence of antioxidants can inhibit the formation of ABTS+. Therefore, the lower the absorbance measured at 734 nm, the greater anti-oxidation ability of the antioxidant.

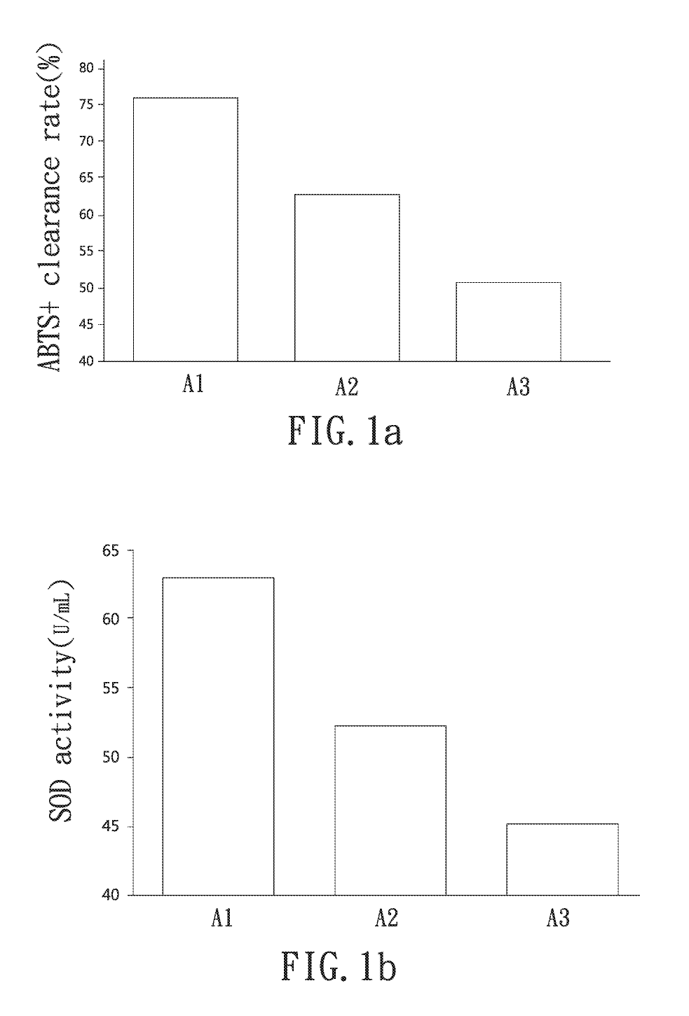

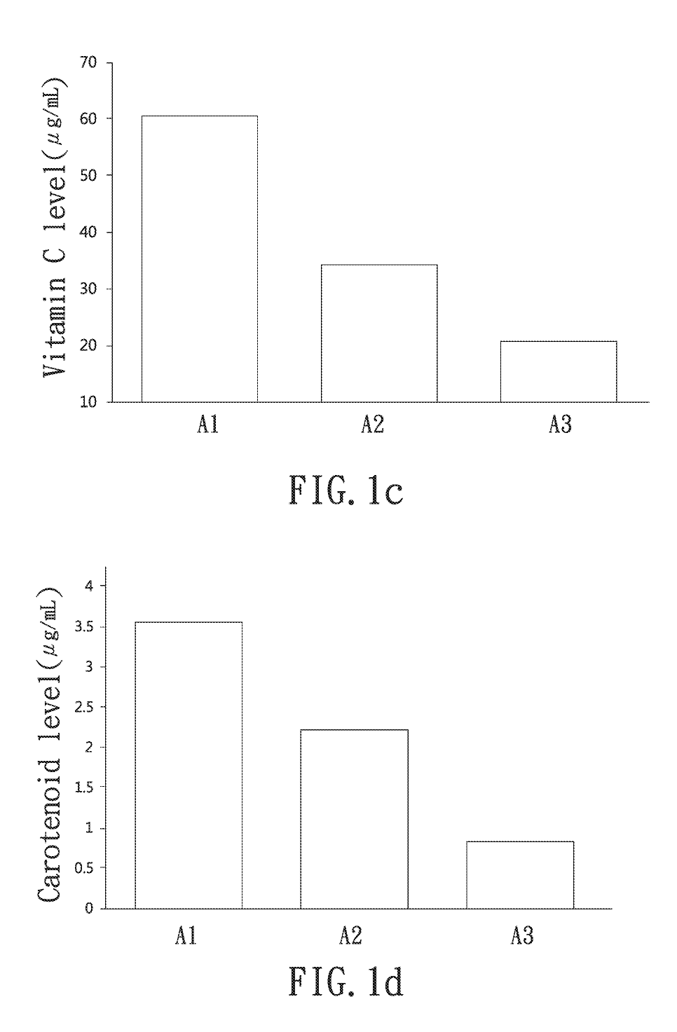

Accordingly, 500-fold diluted fermented juices of groups A1 & A2 as well as 500-fold diluted unfermented juice of group A3 are used for measuring the ABTS+ clearance rate (%). Referring to FIG. 1a, compared to the fermented juice of group A2, which is obtained by fermenting using the another microbial mixture containing the L. acidophilus strain, the B. longum strain and the S. cerevisiae strain, and to the unfermented juice of group A3, the fermented juice of group A1 obtained by fermenting the fruit sample by the microbial mixture containing the L. casei strain, the B. longum strain and the S. cerevisiae strain has a highest ABTS+ clearance rate (%).

100-fold diluted fermented juices of groups A1 & A2 as well as 100-fold diluted unfermented juice of group A3 are used for measuring the superoxidase dismutase (SOD) activity. Referring to FIG. 1b, compared to the fermented juice of group A2, which is obtained by fermenting using the another microbial mixture containing the L. acidophilus strain, the B. longum strain and the S. cerevisiae strain, and to the unfermented juice of group A3, the fermented juice of group A1 obtained by fermenting the fruit sample by the microbial mixture containing the L. casei strain, the B. longum strain and the S. cerevisiae strain has a relative higher SOD activity.

500-fold diluted fermented juices of groups A1 & A2 as well as 500-fold diluted unfermented juice of group A3 are used for measuring vitamin C level and carotenoid level. Referring to FIGS. 1c & 1d, compared to the fermented juice of group A2 and the unfermented juice of group A3, the fermented juice of group A1 has significant higher vitamin C level and carotenoid level. That is, both vitamin C level and carotenoid level increase in the fermented juice of group A1, which is obtained by fermenting using the microbial mixture containing the L. casei strain, the B. longum strain and the S. cerevisiae strain.

In conclusion, compared to not only the fruit sample without fermentation but also to the fermented juice obtained by fermenting the fruit sample by the another microbial mixture containing the L. acidophilus strain, the B. longum strain and the S. cerevisiae strain, the fermented juice, obtained by fermenting the fruit sample by the microbial mixture containing the L. casei strain, the B. longum strain and the S. cerevisiae strain, has improved anti-oxidation ability and increased antioxidant levels. That is, by using the method for fermenting Rosa sterilis var. leioclada according to the present invention, the fermented juice with improved anti-oxidation ability can be obtained.

Trial (B).

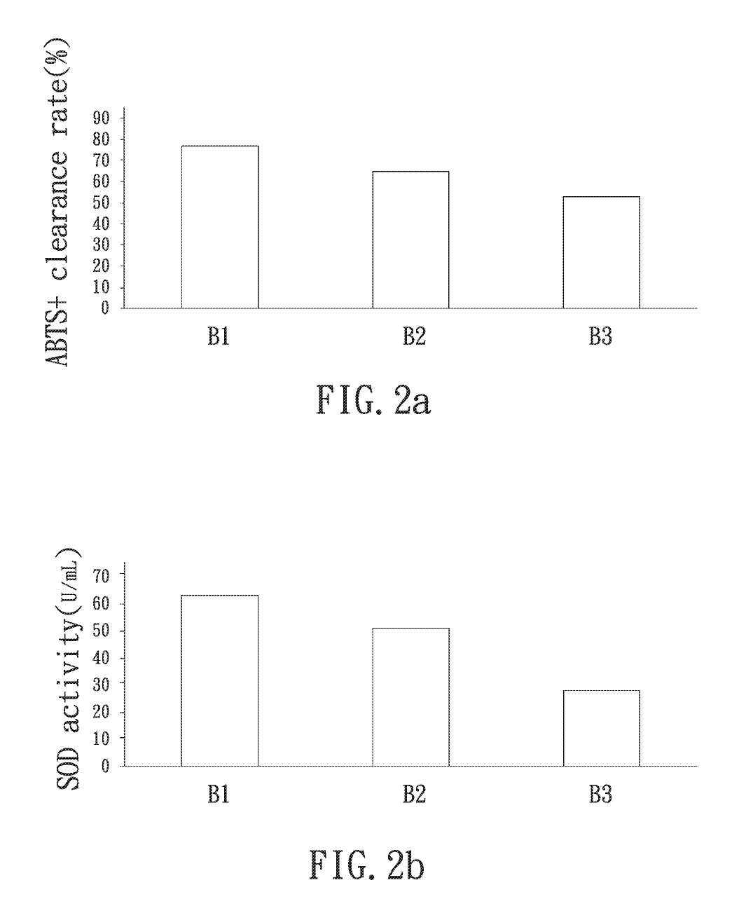

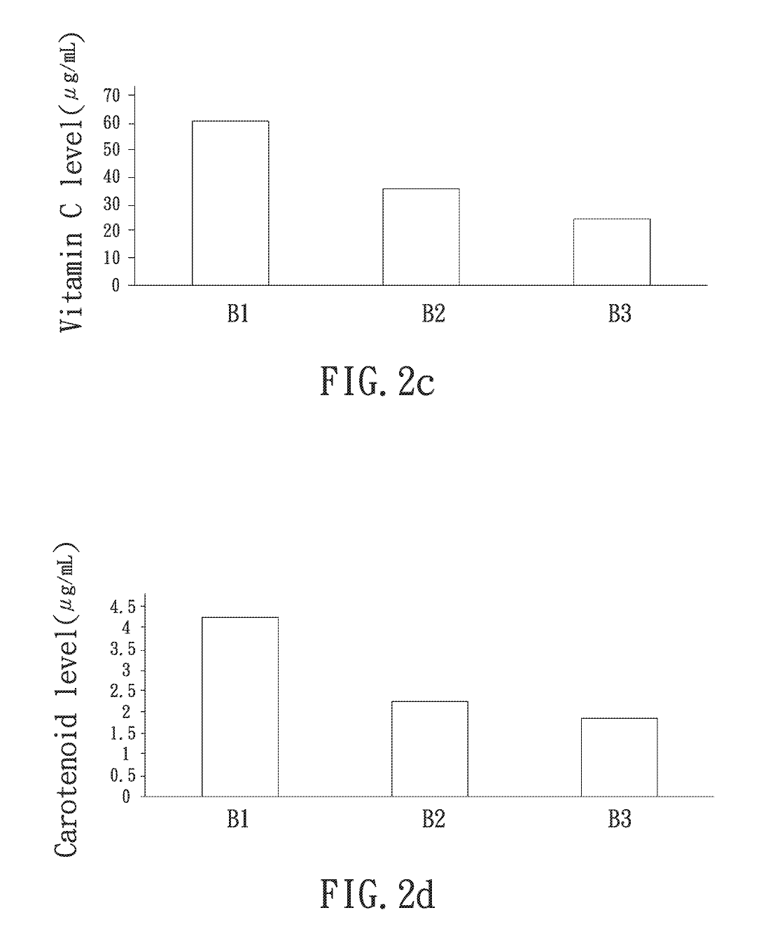

In trial (B), the L. casei strain, the B. longum strain and the S. cerevisiae strain are mixed in the cell number ratio of 0.8:0.8:1.6 to obtain the microbial mixture. The microbial mixture and the juice are mixed to obtain the mixture solution with the total concentration of the L. casei strain, the B. longum strain and the S. cerevisiae strain in the mixture solution being 3×10⁷ CFU/mL. The juice is then fermented by the microbial mixture at 28° C. for 14 days to form the fermented juice of group B1. In addition, the juice is fermented by the microbial mixture with the total concentration of the L. casei strain, the B. longum strain and the S. cerevisiae strain in the mixture solution being 1×10⁷ CFU/mL to form the fermented juice of group B2. The unfermented juice is used as group B3. Anti-oxidation ability (ABTS+ clearance rate and SOD activity), as well as antioxidants (vitamin C and carotenoid) level of the fermented juices of groups B1 & B2 and the unfermented juice of group B3 are measured. Referring to FIGS. 2a and 2b, the fermented juice of group B1 has significantly improved ABTS+ clearance rate and significantly increased SOD activity. Moreover, as shown in FIGS. 2c and 2d, the fermented juice of group B1 also has elevated antioxidant level such as vitamin C level and carotenoid level. That is, compared to fermenting the fruit sample by the microbial mixture with the total concentration of the L. casei strain, the B. longum strain and the S. cerevisiae strain in the mixture solution being 1×10⁷ CFU/mL, fermenting the fruit sample by the microbial mixture with the total concentration of the L. casei strain, the B. longum strain and the S. cerevisiae strain in the mixture solution being 3×10⁷ CFU/mL can effectively obtain the fermented juice with improved anti-oxidation ability.

Trial (C).

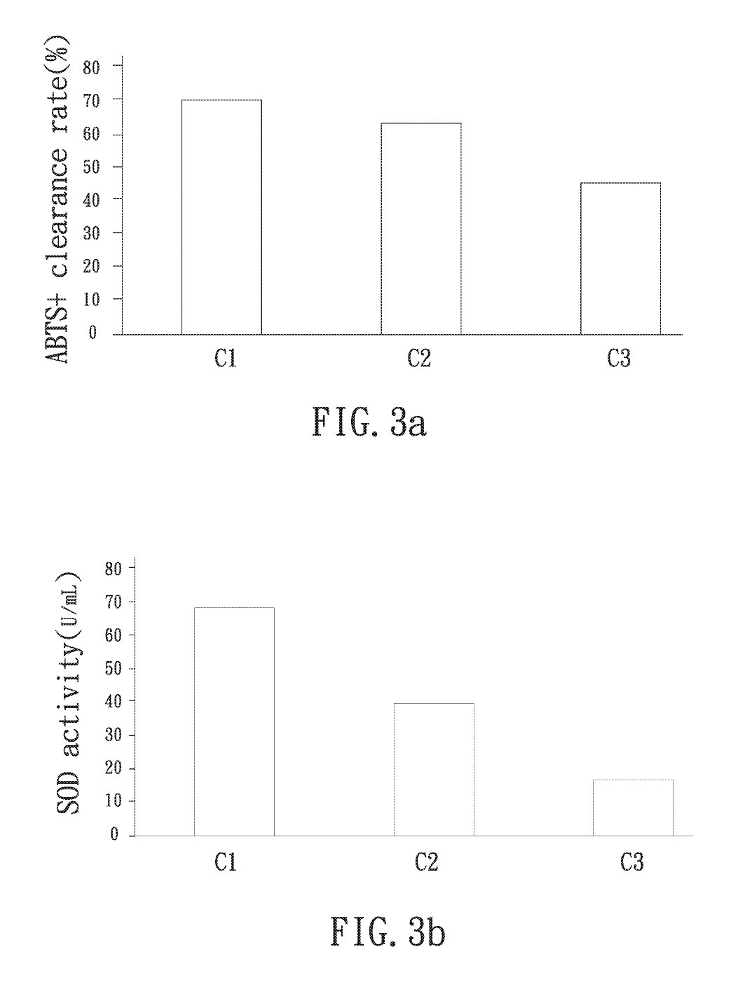

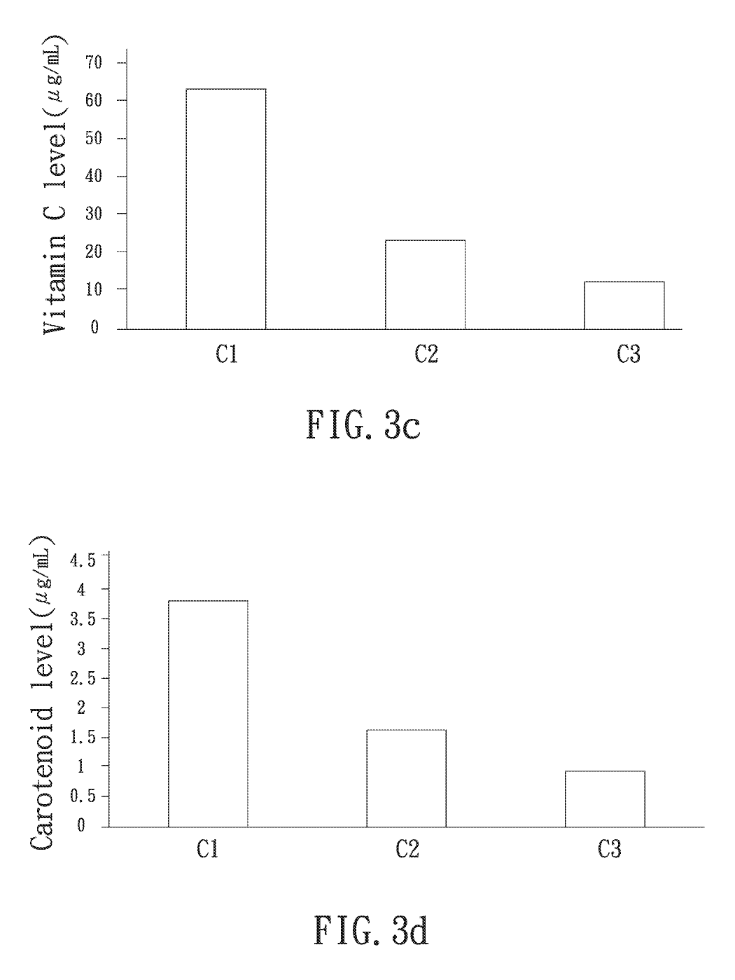

In trial (C), the L. casei strain, the B. longum strain and the S. cerevisiae strain are mixed in the cell number ratio of 0.8:0.8:1.6 to obtain the microbial mixture with. The microbial mixture and the juice are mixed to obtain the mixture solution with the total concentration of the L. casei strain, the B. longum strain and the S. cerevisiae strain in the mixture solution being 3×10⁷ CFU/mL. The juice is then fermented by the microbial mixture at 28° C. for 14 days to form the fermented juice of group C1. In addition, the juice is fermented by the microbial mixture at 20° C. for 14 days to form the fermented juice of group C2. The unfermented juice is used as group C3. Anti-oxidation ability (ABTS+ clearance rate and SOD activity), as well as antioxidants (vitamin C and carotenoid) level of the fermented juices of groups C1 & C2 and the unfermented juice of group C3 are measured. Referring to FIGS. 3a and 3b, the fermented juice of group C1 has significantly improved ABTS+ clearance rate and significantly increased SOD activity. Moreover, as shown in FIGS. 3c and 3d, the fermented juice of group C1 also has elevated antioxidant level such as vitamin C level and carotenoid level. That is, compared to fermenting the fruit sample at 20° C., fermenting the fruit sample at 28° C. can effectively obtain the fermented juice with improved anti-oxidation ability.

Trial (D).

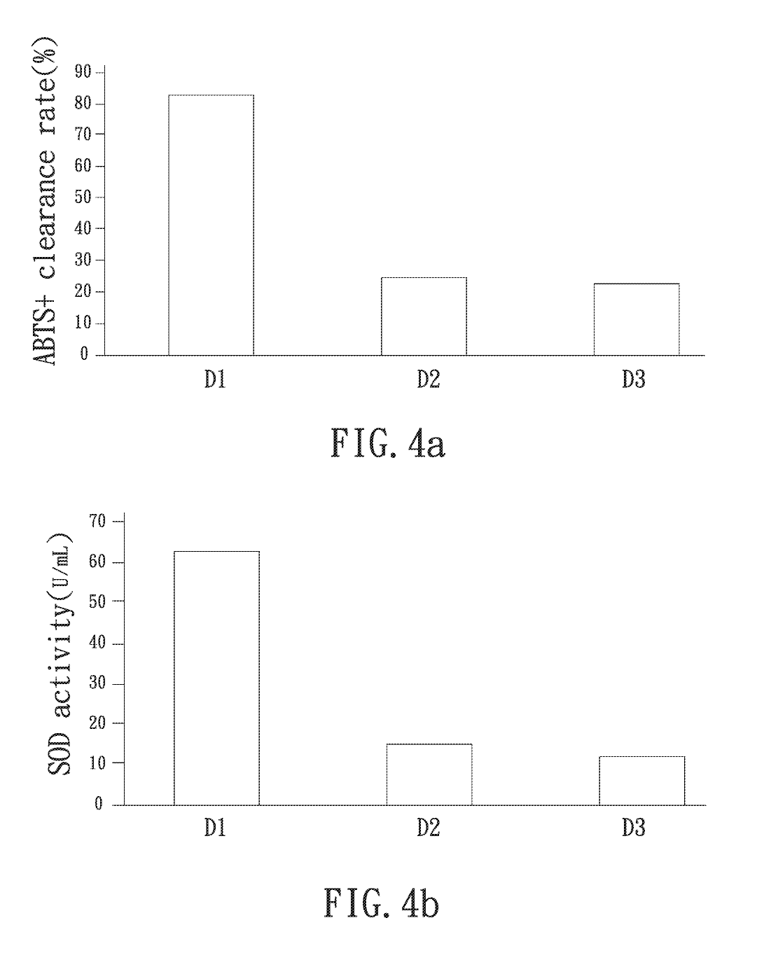

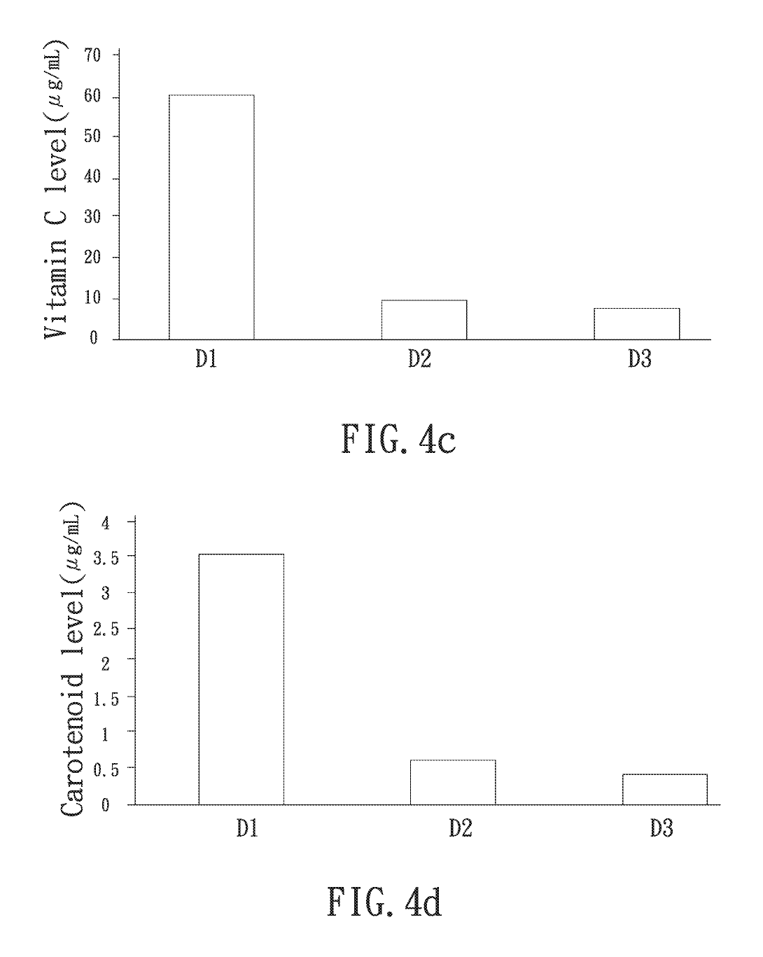

In trial (D), the L. casei strain, the B. longum strain and the S. cerevisiae strain are mixed in the cell number ratio of 0.8:0.8:1.6 to obtain the microbial mixture. The microbial mixture and the juice are mixed to obtain the mixture solution with the total concentration of the L. casei strain, the B. longum strain and the S. cerevisiae strain in the mixture solution being 3×10⁷ CFU/mL. The juice is then fermented by the microbial mixture at 28° C. for 14 days to form the fermented juice of group D1. In addition, the juice is fermented by the microbial mixture at 28° C. for 5 days to form the fermented juice of group D2. The unfermented juice is used as group D3. Anti-oxidation ability (ABTS+ clearance rate and SOD activity), as well as antioxidants (vitamin C and carotenoid) level of the fermented juices of groups D1 & D2 and the unfermented juice of group D3 are measured. Referring to FIGS. 4a and 4b, the fermented juice of group D1 has significantly improved ABTS+ clearance rate and significantly increased SOD activity. Moreover, as shown in FIGS. 4c and 4d, the fermented juice of group D1 also has elevated antioxidant level such as vitamin C level and carotenoid level. Moreover, the fermented juice of group D2 shows similar anti-oxidation ability and antioxidants level to the unfermented juice of group D3. That is, compared to fermenting the fruit sample for 5 days, fermenting the fruit sample for 14 days can effectively obtain the fermented juice with improved anti-oxidation ability.

In conclusion, by fermenting the fruit sample by the microbial mixture including the L. casei strain, the B. longum strain and the S. cerevisiae strain, the obtained fermented juice has improved anti-oxidation ability. Therefore, the fermented juice can be used as a healthy product as well as an additive with anti-oxidation ability.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A method for fermenting Rosa sterilis var. leioclada, comprising:
providing a fruit sample of Rosa sterilis var. leioclada;
mixing a Lactobacillus casei strain, a Bifidobacterium longum strain and a Saccharomyces cerevisiae strain to form a microbial mixture, wherein a cell number ratio of the Lactobacillus casei strain, the Bifidobacterium longum strain and the Saccharomyces cerevisiae strain is 0.3-1:0.2-0.9:1-1.8;
fermenting the fruit sample of Rosa sterilis var. leioclada by the microbial mixture at 22-33° C. for 6-15 days to obtain a fermented juice, wherein a total concentration of the Lactobacillus casei strain, the Bifidobacterium longum strain and the Saccharomyces cerevisiae strain in a mixture solution containing the microbial mixture and the fruit sample of Rosa sterilis var. leioclada is between 1.5×10⁷ and 3.5×10⁷ CFU/mL.

2. The method for fermenting Rosa sterilis var. leioclada as claimed in claim 1, wherein a Lactobacillus casei strain BCRC 10697, a Bifidobacterium longum strain BCRC 14604 and a Saccharomyces cerevisiae strain BCRC 20579 are mixed to form the microbial mixture.

3. The method for fermenting Rosa sterilis var. leioclada as claimed in claim 1, wherein the fruit sample of Rosa sterilis var. leioclada is fermented by the microbial mixture at 28° C.

4. The method for fermenting Rosa sterilis var. leioclada as claimed in claim 1, wherein the fruit sample of Rosa sterilis var. leioclada is fermented by the microbial mixture for 14 days.
